SEQUENCE LISTING (17 sequences)

SEQ ID NO: 1 - protein, 5 aa, Mus musculus
SGGMS

SEQ ID NO: 2 - protein, 17 aa, Mus musculus
TISNGGGETYYEDSVKG

SEQ ID NO: 3 - protein, 11 aa, Mus musculus
EEYEGTWYEDV

SEQ ID NO: 4 - protein, 16 aa, Mus musculus
TSSQSGVHSNGNTELE

SEQ ID NO: 5 - protein, 7 aa, Mus musculus
KVSNRTS

SEQ ID NO: 6 - protein, 9 aa, Mus musculus
GQGSHVGRT

SEQ ID NO: 7 - protein, 137 aa, Artificial Sequence
  heavy chain variable region
MNFGLSLIFLALILKGVQCEVQLVESGGDLVKPGGSLKLSCAASGFTFSSGGMSWVRQTPDKRLEWATISNGGGETYYEDSVKGRFTISRDNAKNTLYLQMNSLKSEDTAMYYCAREEYEGTWYEDVWGAGTVTVSS

SEQ ID NO: 8 - protein, 129 aa, Artificial Sequence
  light chain variable region
MKLPVRLLVLMFWIPASSSDVLMTQTPLSLPVSLGDQASISCTSSQSGVHSNGNTELEWYLQKPGQPKLLIYKVSNRTSGVPDRFSGSGSGTDFTLKISRVEAEDLGVYYCGQGSHVGRTFGGGTKLEI

SEQ ID NO: 9 - protein, 14 aa, Artificial Sequence
  polypeptide
DSVNVDKQPKYEYS

SEQ ID NO: 10 - DNA, 417 bp, Artificial Sequence
  VH
atgaacttcgggctcagcttgattttccttgccctcattttaaaaggtgtccagtgtgaggtgcagctggtggagtctgggggagacttagtgaagcctggagggtccctgaaactctcctgtgcagcctctggattcactttcagtagcggcggcatgtcttgggttcgccagactccagacaagaggctggagtgggtcgcaaccattagtaatggtggtggtgagacctactatgaggacagtgtgaaggggcgattcaccatctccagagacaatgccaagaacaccctgtacctacaaatgaacagtctgaagtctgaggacacagccatgtattactgtgctagggaggagtacgagggtacgtggtactgagatgtctggggcgcagggaccacggtcaccgtctcctca

SEQ ID NO: 11 - DNA, 393 bp, Artificial Sequence
  VL
atgaagttgcctgttaggctgttggtgctgatgttctggattcctgcttccagcagtgatgttttgatgacccaaactccactctccctgcctgtcagtcttggagatcaagcctccatctcttgcacatctagtcagagcggagtacatagtaatggaaacaccgagttagaatggtacctgcagaaaccaggccagtctccaaagctcctgatctacaaagtttccaaccgaacatctggggtcccagacaggttcagtggcagtggatcagggacagatttcacactcaagatcagcagagtggaggctgaggatctgggagtttattactgcggccaaggttcacatgttggccggacgttcggtggaggcaccaagctggaaatcaaa

SEQ ID NO: 12 - RNA, 417 nt, Artificial Sequence
  RNA sequence encoding VH
augaacuucgggcucagcuugauuuuccuugcccucauuuuaaaagguguccagugugaggugcagcugguggagucugggggagacuuagugaagccuggagggucccugaaacucuccugugcagccucuggauucacuuucaguagcggcggcaugucuuggguucgccagacuccagacaagaggcuggagugggucgcaaccauuaguaauggugguggugagaccuacuaugaggacagugugaaggggcgauucaccaucuccagagacaaugccaagaacacccuguaccuacaaaugaacagucugaagucugaggacacagccauguauuacugugcuagggaggaguacgaggguacgugguacugagaugucuggggcgcagggaccacggucaccgucuccuca

SEQ ID NO: 13 - RNA, 393 nt, Artificial Sequence
  RNA sequence encoding VL
augaaguugccuguuaggcuguuggugcugauguucuggauuccugcuuccagcagugauguuuugaugacccaaacuccacucucccugccugucagucuuggagaucaagccuccaucucuugcacaucuagucagagcggaguacauaguaauggaaacaccgaguuagaaugguaccugcagaaaccaggccagucuccaaagcuccugaucuacaaaguuuccaaccgaacaucuggggucccagacagguucaguggcaguggaucagggacagauuucacacucaagaucagcagaguggaggcugaggaucugggaguuuauuacugcggccaagguucacauguuggccggacguucgguggaggcaccaagcuggaaaucaaa

SEQ ID NO: 14 - protein, 456 aa, Artificial Sequence
  heavy chain
MNFGLSLIFLALILKGVQCEVQLVESGGDLVKPGGSLKLSCAASGFTFSSGGMSWVRQTPDKRLEWATISNGGGETYYEDSVKGRFTISRDNAKNTLYLQMNSLKSEDTAMYYCAREEYEGTWYEDVWGAGTAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 236 aa, Artificial Sequence
  light chain
MKLPVRLLVLMFWIPASSSDVLMTQTPLSLPVSLGDQASISCTSSQSGVHSNGNTELEWYLQKPGQPKLLIYKVSNRTSGVPDRFSGSGSGTDFTLKISRVEAEDLGVYYCGQGSHVGRTFGGGTKLEIKRAAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 1392 bp, Artificial Sequence
  encoding the heavy chain
atgaacttcgggctcagcttgattttccttgccctcattttaaaaggtgtccagtgtgaggtgcagctggtggagtctgggggagacttagtgaagcctggagggtccctgaaactctcctgtgcagcctctggattcactttcagtagcggcggcatgtcttgggttcgccagactccagacaagaggctggagtgggtcgcaaccattagtaatggtggtggtgagacctactatgaggacagtgtgaaggggcgattcaccatctccagagacaatgccaagaacaccctgtacctacaaatgaacagtctgaagtctgaggacacagccatgtattactgtgctagggaggagtacgagggtacgtggtactgagatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 17 - DNA, 717 bp, Artificial Sequence
  encoding the light chain
atgaagttgcctgttaggctgttggtgctgatgttctggattcctgcttccagcagtgatgttttgatgacccaaactccactctccctgcctgtcagtcttggagatcaagcctccatctcttgcacatctagtcagagcggagtacatagtaatggaaacaccgagttagaatggtacctgcagaaaccaggccagtctccaaagctcctgatctacaaagtttccaaccgaacatctggggtcccagacaggttcagtggcagtggatcagggacagatttcacactcaagatcagcagagtggaggctgaggatctgggagtttattactgcggccaaggttcacatgttggccggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

(19) United States
(12) Patent Application Publication
(10) Pub. No.: US 2025/0277027 A1
(43) Pub. Date: Sep. 4, 2025

(54) ANTIBODY OR ANTIBODY FRAGMENT THAT SPECIFICALLY BINDS TO VOLTAGE-GATED SODIUM CHANNEL ALPHA SUBUNIT NAV1.7

(71) Applicant: POPULAS BIOPHARMACEUTICAL(WUHAN) LIMITED, Wuhan (CN)

(72) Inventor: Daichang YANG, Wuhan (CN)

(21) Appl. No.: 18/562,807

(22) PCT Filed: May 21, 2021
(86) PCT No.: PCT/CN2021/095059

§ 371 Date: Nov. 20, 2023

(51) Int. Cl. C07K 16/28; A61K 39/00

(52) U.S. Cl. CPC C07K 16/28; A61K 2039/505; C07K 2317/33; C07K 2317/92

(57) ABSTRACT

The present invention provides an antibody or antibody fragment that targets cell membrane voltage-gated sodium ion channel α subunit Nav 1.7; a specific binding target thereof is an ion-conducting pore module (PM) of an S3 domain of domain IV of a voltage-gated sodium ion channel α subunit. The antibody or antibody fragment thereof can inactivate the ion-conducting PM, so that sodium ions cannot normally enter nerve cells, to thereby achieve the effect of treating and relieving pain.

### FIELD OF THE INVENTION

The present invention belongs to the field of biomedicine and relates to an antibody and/or antibody fragment that targets the ion-conducting pore module of Nav1.7 and specifically recognizes the target (polypeptide).

### REFERENCE TO SEQUENCE LISTING SUBMITTED VIA EFS-WEB

This application includes an electronically submitted sequence listing in .txt format. The .txt file contains a sequence listing entitled “WH1917-23P151186US ST25.txt” created on Nov. 16, 2023 and is 15,306 bytes in size. The sequence listing contained 20 in this .txt file is part of the specification and is hereby incorporated by reference herein in its entirety.

### BACKGROUND OF THE INVENTION

Pain begins with the nociceptors of the peripheral nerve system, and peripheral nerve tissues are widely distributed in the skin, muscles, joints and visceral tissues of the whole body as a kind of free nerve ending, and can convert thermal, mechanical or chemical stimuli into action potentials, transmit them to the cell body in the dorsal root ganglia (DRG) through nerve fibers and ultimately to the advanced nerve center, thereby causing pain. The generation and conduction of action potentials in neurons in turn depend on the voltage-gated sodium channels (VGSCs) located on the cytomembrane. When the cytomembrane is depolarized, the sodium ion channel is activated. The channel is opened, causing sodium ion influx, and further depolarizing the cytomembrane, resulting in the generation of an action potential, and thus causing pain due to the abnormal action potential. Therefore, inhibition of abnormal sodium ion channel activity contributes to the treatment and alleviation of pain.

Voltage-gated sodium channels can be categorized into nine subtypes. At present, nine a-subtypes of voltage-gated sodium channels have been identified in mammals, since their amino acid sequences have more than 50% similarity, they are considered to be from the same family, named Nav1 (Nav1.1-Nav1.9). Voltage-gated sodium channels, widely found on the cytomembranes of neuron and skeletal muscle cells, are a class of transmembrane glycoprotein complexes composed of an α subunit and a number of auxiliary β subunits. The α-subunit is composed of two functional domains, i.e. an ion-conducting pore domain and voltage-sensing domains (VSDs). The pore of the α-subunit consists of four repeating domains (DI-DIV), each of which contains six transmembrane helical fragments (S1-S6). S1-S4 contain the voltage-sensing domain VSD, and S5-S6 form a tetrameric conformation to form the pore domain. Within the VSD domain, S4 contains the VSD, and S4 is enriched in gating-charge arginine, which senses changes in membrane potential. Together with the C-terminus of S3, it forms a voltage-sensor paddle, whose movement reflects changes in membrane potential and is coupled to pore opening, closing, and inactivation. Since this voltage-sensor paddle movement is the opening and locking of the channel. Therefore, this functional structural domain is an important drug action target and can be used through protein interactions to regulate the switching of channels.

Recent studies have shown that the main subtypes of Nav1 associated with pain are Nav1.7, Nav1.8, and Nav1.9. Among them, Nav1.7 is one of the important members mainly responsible for pain. Nav1.7 is a TTX-S type and encodes the gene SCN9 A. It is mainly distributed in peripheral primary sensory neurons and sympathetic ganglion neurons, and is involved in the human pain signaling pathway. Recently, in human pain-free patients, pain-free symptoms have occurred following genetic mutations in Nav1.7; further studies have shown that this gene is one of the sodium ion channels primarily responsible for pain.

Small chemical molecules (e.g., carbamazepine, lidocaine, and mexiletine) are commonly used clinically as voltage-gated sodium channel inhibitors for the treatment of pain; however, they lack sufficient selectivity for voltage-gated sodium channel subtypes, and thus have the disadvantage of cardiotoxicity and central nervous system side effects. Recently, some small-molecule blockers against Nav1.7 have been introduced into clinical studies, but due to the high homology of sodium channel subtypes, small-molecule blockers have poor selectivity and their side effects are difficult to overcome. Large molecule blockers have high specificity, good stability, and fewer side effects, but they are difficult to study because the antibody-producing voltage-gated sodium channel antigen is not easy to prepare.

### SUMMARY OF THE INVENTION

An object of the present invention is to provide an antibody or an antibody fragment that specifically binds to the voltage-gated sodium channel α-subunit Nav1.7. The specific binding means that the ion-conducting pore module (PM) of the DIVS3 domain of Nav1.7 is used as a target, the target in this region is used to design a polypeptide as an antigen to obtain a monoclonal antibody, and the specific antibody can interfere with the normal state of the VGSCs through binding to its target, thereby inhibiting pain.

A second object of the present invention is to provide a pharmaceutical composition comprising the antibody or antibody fragment thereof that specifically binds to the ion-conducting pore module of the DIVS3 domain of the voltage-gated sodium channel α-subunit Nav1.7.

A third object of the present invention is to provide uses of the antibody or antibody fragment thereof that specifically binds to the ion-conducting pore module of the DIVS3 domain of voltage-gated sodium channel α-subunit Nav1.7, or of the pharmaceutical composition.

The present invention also provides a nucleotide encoding the antibody or antibody fragment, an expression vector containing the nucleotide, and a method of preparing the antibody or antibody fragment.

According to an aspect of the present invention, an antibody or an antibody fragment thereof that specifically binds to the ion-conducting pore module of the DIVS3 domain of the voltage-gated sodium channel α-subunit Nav1.7 of the present invention, the target of specific binding is the ion-conducting pore module of the DIV/S3 domain of the voltage-gated sodium channel α-subunit. More preferably, the amino acid sequence of the bound antigen is: DSVNVDKQPKYEYS (SEQ ID NO.9).

Based on the crystal structure of Nav1.7, the ion-conducting pore module of the DIV/S3 domain of the voltage sensor valve of Nav1.7 was used to screen suitable polypeptides in the target region as an antigen, and after the hydrophilicity and antigenicity analysis, a polypeptide with good hydrophilicity and high antigenicity was selected, having the amino acid sequence of DSVNVDKQPKYEYS (SEQ ID NO.9).

The above polypeptide was chemically synthesized, the synthesized polypeptide was numbered C9797BL020-7 (SEQ ID NO. 9), coupled to the carrier protein KLH, and then immunized to BALB/c mice. The body was stimulated by multiple immunizations to produce an immune response, which resulted in a polyclonal antibody for blood collection, ELISA assay and evaluation.

Based on the antigen-antibody reaction, the potency of the polyclonal antibody produced by the immunized animals was evaluated by ELISA. Based on the antibody potency of the immunized animals and the specificity of the human neural tissues, three animals that met the requirements, #4061, #4062 and #4063, were finally identified for cell fusion. The spleen cells of the three animals were electrofused with mouse myeloma cells (SP2/0) and then subjected to cell culture after fusion. The positive cell lines were screened on screening medium, and hybridoma cell lines were screened by using the polypeptide C9797BL020-7 as an antigen. Based on the ELISA results, positive cell lines were selected for subcloning based on antibody potency and human neural tissue specificity. The resulting subclones were again subjected to ELISA and neural tissue specificity assays, and those with good specificity for nerve tissues were selected for subcloning for cell cryopreservation.

Total RNA of the cell line was extracted, cDNA was synthesized, a cDNA library was created, and variable regions were sequenced. The polynucleotide sequences encoding the variable regions of the antibody were amplified, and the DNA sequences encoding VH and VL (which could also be manipulated with RNA sequences encoding the variable regions) could be integrated into the same vector, or they could be integrated into the vector separately, and suitable host cells were transfected with the above vector; and then sequenced and analyzed. The sequencing results showed that the VH has the DNA sequence as shown in SEQ ID NO: 10 and the VL has the DNA sequence as shown in SEQ ID NO: 11.

Genetically engineered antibodies were constructed. According to different needs, the above DNA sequences encoding VH and VL (or encoding CDR in VH and encoding CDR in VL) were introduced into a suitable host for antibody expression, and the antibody effect was verified.

The immunogenicity of the monoclonal antibody was tested. The target sequence was prokaryotically expressed, and the total protein was extracted from the prokaryotically expressed bacteria, and then subjected to preliminary purification to obtain an antigenic fragment. The binding specificity of the antibody was analyzed by Western Blotting, as shown in FIG. 4, the antibody can specifically recognize the target protein sequence of Nav1.7. An acute inflammatory pain mice model was established by treating with 5% formalin. An appropriate amount of antibody was injected via tail vein to detect the analgesic effect of the antibody on the pain model mice. The results as shown in FIG. 5, showed that the injection of 10 mg/kg of antibody had a significant analgesic effect over the control.

According to another aspect of the present invention, an antibody or an antibody fragment thereof that specifically binds to the voltage-gated sodium channel α subunit Nav1.7, comprises:
heavy chain complementary determining regions HCDR1, HCDR2, HCDR3, the HCDR1 having the amino acid sequence as shown in SEQ ID NO. 1, the HCDR2 having the amino acid sequence as shown in SEQ ID NO. 2, the HCDR3 having the amino acid sequence as shown in SEQ ID NO. 3; and
light chain complementary determining regions LCDR1, LCDR2, LCDR3, the LCDR1 having the amino acid sequence as shown in SEQ ID NO. 4, the LCDR2having the amino acid sequence as shown in SEQ ID NO. 5, the LCDR3 having the amino acid sequence as shown in SEQ ID NO. 6.

According to the present invention, the antibody is a monoclonal antibody or a polyclonal antibody. Preferably, the antibody is a monoclonal antibody.

According to the present invention, the antibody is a murine antibody, a chimeric antibody or a humanized antibody, etc. Preferably, the antibody is a humanized antibody.

According to the present invention, the antibody fragment comprises the forms of Fab, F(ab′)2, dsFv, scFv, a diabody, a minibody, a bispecific antibody, a multi-specific antibody, a chimeric antibody, and a CDR-grafted antibody.

According to a preferred embodiment of the present invention, the antibody or antibody fragment that specifically binds to the voltage-gated sodium channel α subunit Nav1.7 comprises a heavy chain variable region and a light chain variable region. The heavy chain variable region has the amino acid sequence as shown in SEQ ID NO. 7, and the light chain variable region has the amino acid sequence as shown in SEQ ID NO. 8. It will be appreciated by those skilled in the art that the antibody or antibody fragment of the present invention also comprise a structurally similar derivative sequence with 80%, 80-85%, 85-90%, 90-95% or 95-99% homology to the sequence of SEQ ID NO: 7/SEQ ID NO.8 of the heavy/light chain variable region.

According to a preferred embodiment of the present invention, the antibody or antibody fragment that specifically binds to the voltage-gated sodium channel α subunit Nav1.7 comprises a heavy chain constant region and a light chain constant region. The species sources of the light chain constant region and the heavy chain constant region may be selected from the group consisting of human antibody constant region, bovine antibody constant region, sheep antibody constant region, canine antibody constant region, porcine antibody constant region, feline antibody constant region, equine antibody constant region, and donkey antibody constant region. Preferably, the heavy chain constant region is selected from the group consisting of IgG1, IgG2, IgG3 and IgG4 heavy chain constant regions and the light chain constant region is selected from the group consisting of κ or λ light chain constant region. Preferably, the heavy chain constant region is an IgG4 heavy chain constant region and the light chain constant region is a κ light chain constant region.

According to a preferred embodiment of the present invention, the antibody or antibody fragment that specifically binds to the voltage-gated sodium channel α subunit Nav1.7 comprises a heavy chain and a light chain. The heavy chain has the amino acid sequence of SEQ ID NO: 14 and the light chain has the amino acid sequence of SEQ ID NO: 15.

According to a further aspect of the present invention, provided is a nucleotide sequence. The nucleotide sequence encodes the antibody or antibody fragment that specifically binds to the voltage-gated sodium channel α subunit Nav1.7 of the present invention.

According to a preferred embodiment of the present invention, the nucleotide sequence comprises: a nucleotide sequence encoding the heavy chain variable region as shown in SEQ ID NO: 10, a nucleotide sequence encoding the light chain variable region as shown in SEQ ID NO: 11.

According to a preferred embodiment of the present invention, the nucleotide sequence comprises: a nucleotide sequence encoding the heavy chain as shown in SEQ ID NO: 16, a nucleotide sequence encoding the light chain as shown in SEQ ID NO: 17.

The present invention also provides an expression vector, which contains anyone of the nucleotide sequence described above.

The present invention also provides a host cell, which contains the expression vector described above.

The present invention also provides a method of preparing the antibody or antibody fragment that specifically binds to the voltage-gated sodium channel α subunit Nav1.7 described above, the method comprises the following steps of:
(a) culturing the host cell described above under expression conditions so as to express the antibody or antibody fragment that specifically binds to the voltage-gated sodium channel α subunit Nav1.7;
(b) isolating and purifying the antibody or antibody fragment that specifically binds to the voltage-gated sodium channel α subunit Nav1.7 of (a).

According to another aspect of the present invention, provided is a pharmaceutical composition. The pharmaceutical composition comprises the antibody or antibody fragment that specifically binds to the voltage-gated sodium channel α subunit Nav1.7 described herein as an active ingredient, and a pharmaceutically acceptable carrier. The pharmaceutical composition has an analgesic and pain threshold raising effect and can treat pain, itching and cough.

According to a further aspect of the present invention, provided is the use of the antibody or antibody fragment that specifically binds to the voltage-gated sodium channel α subunit Nav1.7 described herein or a pharmaceutical composition described herein in the manufacture of a medicament for the treatment of pain-related diseases.

Beneficial effects: The specific binding of the antibody, a biomolecule targeted against the voltage sensor of the Nav1.7 voltage-gated sodium channel, is used to inactivate the ion-conducting pore module of the DIVS3 domain, so that sodium ions cannot normally enter nerve cells, thus achieving the effect of treating and relieving pain. Due to its excellent targeting properties, it can overcome the side effects due to chemical small molecule drugs.

### DESCRIPTION OF THE DRAWINGS

FIG. 1: Structural diagram of the sodium channel Nav1.7 and schematic of the target;

FIG. 2: Immunohistochemical analysis of human neural tissue incubated with sera from immunized animals;

A. 4061; B. 4062; C.4063; D. 4064; E. 4065

FIG. 3: Western blotting immunogenicity analysis of monoclonal antibodies;

FIG. 4: Affinity binding curve of monoclonal antibody 5C12D2C8 to Nav1.7 target determined by SPR;

FIG. 5: Localization of monoclonal antibody 5C12D2C8 that targets to Nav1.7 in human peripheral nerve cells;

FIG. 6: Analgesic effect of 5C12D2C8 antibody on 5% formalin-induced acute inflammatory pain in wild-type mice.

### DETAILED DESCRIPTION OF THE INVENTION

The invention is illustrated, but not limited by the following detailed description of the preferred embodiments of the invention.

### Material Sources

The materials and reagents used below are commercially available unless otherwise stated.

### Example 1 Synthesis of Antigen

According to the amino acid sequence (GenBank No. NP_002968) and the functional regions of the crystal structure of Nav1.7 (FIG. 1), hydrophilicity and antigenicity analysis was performed. The sequence of DSVNVDKQPKYEYS was screened, the hydrophilicity and antigenicity of which met the requirements of the antigen. The CDSVNVDKQPKYEYS (SEQ ID NO.9) polypeptide was synthesized using a fully automated synthesizer.

Specific steps were as follows:
(1) attaching —COOH of the first AA to Cl-Resin with DIEA, and then blocking the unreacted functional groups on the resin with MeOH;
(2) washing with DMF;
(3) removing the protecting group Fmoc of —NH₂ in the first AA with Pip to expose the —NH₂;
(4) washing with DMF;
(5) activating —COOH of the second AA with DIC+HOBT, and then condensing it with —NH₂ in the first AA to form an amide bond;
(6) washing with DMF;
(7) removing the protecting group Fmoc of —NH₂ in the second AA with Pip to expose the —NH₂;
(8) washing with DMF;
(9) . . . repeating the steps 5-8 until exposing the —NH₂ of the last AA;
(10) cutting the polypeptide from the resin and removing the side chain protecting groups of all amino acids with the cleavage reagent as: trifluoroacetic acid+ethanedithiol+phenol+thioanisole+water;
(11) adding the cleavage solution into diethyl ether to precipitate the polypeptide, and centrifuging to obtain the crude polypeptide (C9797BL020-7);
(12) purifying the polypeptide with the HPLC C18 preparative/analytical column, designated as C9797BL020-7, to obtain the purified polypeptide for immunizing animals.

Note: To facilitate polypeptide coupling, an additional cysteine may be added to the end of this polypeptide.

### Example 2 Preparation of Monoclonal Cell Lines

### 2.1 Animal Immunization

Freund's complete adjuvant (Sigma, F5881) and Freund's incomplete adjuvant (Sigma, F5506) were prepared. The polypeptide was coupled to the carrier protein KLH by the terminal SH of polypeptide C9797BL020-7, as an immunogen.

Five 8-week-old female BALB/c (animal numbers: #4061, #4062, #4063, #4064, #4065) were selected and immunized intraperitoneally three times to stimulate the body to produce an immune response and then to produce antibodies. Primary immunization: 50 μg/each; the secondary immunization was performed after three weeks, at a dose of 50 μg/each; the third immunization was performed 2 weeks after the second immunization at a dose of 50 μg/each; 1 week after the third immunization, blood was collected for antibody test.

### 2.2 ELISA Test of Animal Serum

### 2.2.1 Instruments and Equipments

Washing machine: Beijing Nanhua ZDMX

Microtiter-plate reader: Thermo Multiskan Ascent

### 2.2.2 Reagents

Coating antigen: polypeptide C9797BL020-7; coating solution: 1*PBS (pH 7.4); washing buffer: 1*PBS (pH 7.4), 0.05% PBS; the primary antibody: anti-serum after the third immunization; enzyme-labeled secondary antibody: Peroxidase-AffiniPure Goat Anti-Mouse IgG, Fcγ Fragment Specific (min X Hu, Bov, HrsSrProt); TMB chromogenic solution; stop solution: 1 M hydrochloric acid.

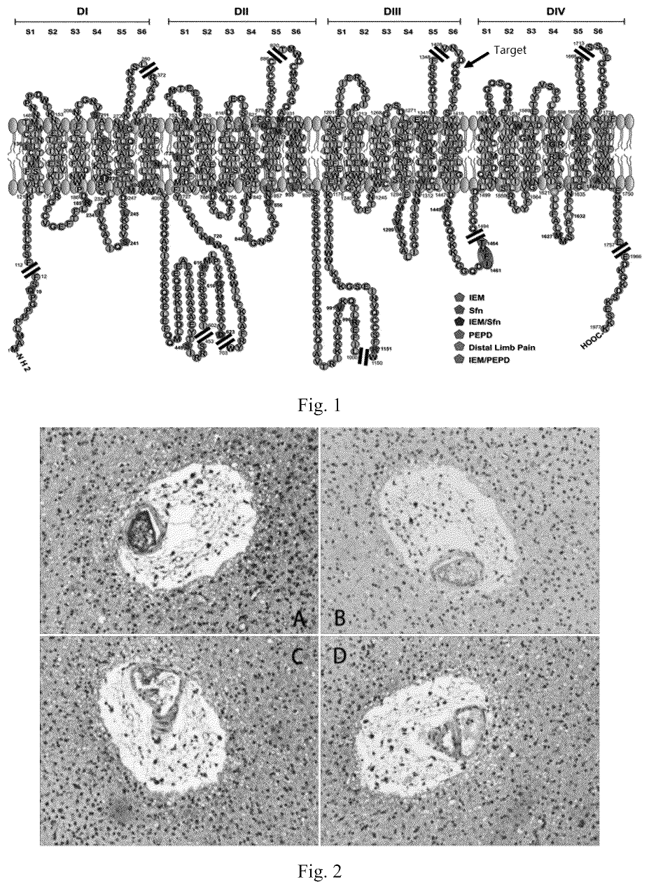

The specific method was as follows:
(1) Coating: The antigen was diluted to 1 μg/ml with the coating solution, mixed and then added to the microtiter-plate at 100 μl per well, covered with the cover film, and placed at 4° C. overnight.
(2) Blocking: The microtiter-plate was taken out to discard the coating solution, added with the blocking solution, covered with the cover film, and incubated at 37° C. for 0.5 h.
(3) Addition of primary antibody: The anti-serum after the third immunization was first diluted 1000-fold, and then subjected to doubling dilution for 9 gradients, covered with the cover film, and incubated at 37° C. for 1 h.
(4) Addition of secondary antibody: The enzyme-labeled microtiter-plate was taken out to discard the solution inside, added with the diluted secondary antibody at a concentration of 0.033 μg/ml, covered with the cover film, and incubated at 37° C. for half an hour.
(5) Color development: The enzyme-labeled microtiter-plate was taken out to discard the solution inside, added with the chromogenic solution to develop the color at 25° C. for 13 min.
(6) Stop of reaction: The stop solution was added to stop the reaction.
(7) The value was read at 450 nm on a microtiter-plate reader immediately after the addition of the stop solution. The maximum dilution corresponding to the well having an OD value of more than 2.1 times the OD value of the set negative control was determined as the titer of the sample, and the test results are as shown in Table 1. NC is a negative control of unimmunized serum, and the initial dilution factor was 1:1,000. The anti-serum after the third immunization was tested, animal Nos. #4061, #4062, and #4063 had antiserum titers at 1:512,000, with the highest S/B values; the remaining two animals (#4064, #4065) had antiserum titers at 1:256,000, but S/B was only 2.7 or less; at the same time the sera were subjected to histochemical analysis of human neuronal cells, and all three mice had a good histochemical signal (FIG. 2). Therefore, three animals were selected for cell fusion.

TABLE 1
Serum ELISA results after third immunization

| Animal No. | #4061 | #4062 | #4063 | #4064 | #4065 |
|---|---|---|---|---|---|
| Blank control | 0.063 | 0.063 | 0.063 | 0.063 | 0.063 |
| 1:1,000 | 2.707 | 2.783 | 2.674 | 2.632 | 2.541 |
| 1:2,000 | 2.606 | 2.58 | 2.532 | 2.438 | 2.405 |
| 1:4,000 | 2.571 | 2.28 | 2.405 | 2.345 | 2.319 |
| 1:8,000 | 2.44 | 2.249 | 2.213 | 1.897 | 1.792 |
| 1:16,000 | 2.206 | 2.01 | 1.934 | 1.624 | 1.435 |
| 1:32,000 | 1.888 | 1.62 | 1.566 | 1.246 | 1.003 |
| 1:64,000 | 1.479 | 1.227 | 1.199 | 0.843 | 0.677 |
| 1:128,000 | 1.087 | 0.833 | 0.826 | 0.529 | 0.403 |
| 1:256,000 | 0.733 | 0.512 | 0.528 | 0.306 | 0.24 |
| 1:512,000 | 0.435 | 0.313 | 0.306 | 0.174 | 0.165 |
| Titer | 1:512,000 | 1:512,000 | 1:512,000 | 1:512,000 | 1:512,000 |
| S/B | 6.905 | 4.968 | 4.857 | 2.762 | 2.619 |

### 2.3 Confirmation of Cytologic Specificity

In order to confirm whether the sera produced by these stimulated immunizations are specific for neural tissues, immunohistochemical analyses of human neural tissues were performed on sera from five animals. The specific experimental methods were as follows.

### 2.3.1 Tissue Dehydration Treatment

The human nerve tissue was taken for dehydration treatment, and the dehydration treatment was performed using Leica ASP300S. The specific process was as follows:

The sections were dehydrated with 70%, 85% and 90% anhydrous ethanol for 30 minutes, respectively; then dehydrated with anhydrous ethanol for 2 times, each time for 60 minutes; then treated with a clearing agent for 30 minutes and then treated with a clearing agent 2 times for 60 minutes each; and then treated with paraffin wax 3 times for 60, 120 and 180 minutes, respectively; then subjected to the embedding operation using a Leica EG1150 embedding machine to make a wax block, which was cut into sections with a thickness of 4 μm.

### 2.3.2 In Situ Hybridization

The human nerve tissue sections were baked at 85° C. for 20 min; treated 3 times with a dewaxing agent for 1 minute each time; dewaxed 3 times with anhydrous alcohol for 1 minute each time; washed 3 times with water for 1 minute each time; thermal repaired with ER2 (pH-9 buffer solution) for 20 minutes, cooled for 12 minutes, then washed 3 times with water for 1 minute each time; then blocked for 30 minutes; washed 3 times with water for 1 minute each time; added with the supernatant of the cell line culture and incubated for 30 minutes, washed 3 times with water for 1 minute each time; incubated for 8 minutes with an enhancer, washed 3 times with water for 2 minutes each time, added with the secondary antibody and incubated for 8 minutes; washed 3 times with water for 2 minutes each time; developed color with DAB for 8 minutes; washed 3 times with water for 1 minute each time, stained with hematoxylin for 10 minutes; washed 3 times with water for 1 minute each time, dehydrated with alcohol, air-dried and sealed. Observations were performed using an Olympus optical microscope.

It was found by optical microscopy that the sera from all five animals reacted positively with human neural tissue, indicating that the immunized animals produced antibodies specific for nerve tissue (FIG. 2).

### 2.4 Cell Fusion

According to the ELISA assay results, combined with the results of human neural tissue specific analysis, three animals #4061, #4062 and #4063 were selected for final immunization, and three days later, the spleen cells of the two animals were fused with tumor cells. The mouse myeloma cells (SP2/0) and spleen cells were electrofused in a 1:3 ratio and the fused cells were plated into 15 feeder cell plates using HAT medium, and cultured in a CO₂ incubator.

### 2.5 Hybridoma Cell Line Screening

After the fusion cells were cultured for 7-10 d, the whole plate was subjected to culture medium change. ELISA assay was performed after 4 h of the medium change. The specific materials and steps of ELISA were the same as those described in 2.2 for ELISA of animal serum.

10 microtiter plates were provided for each animal, a total of 40 96-well plates, a total of 3840 wells, added fusion animal serum with 1:1000 dilution, set as a negative control; added with the blank medium, to detect the OD value of the blank control. The antibody with highest antibody titer and S/B (Signal/Blank)>=2.1 was screened and confirmed as a positive clone. A total of 135 candidate clones were obtained by screening and then used for the next round of subcloning.

### 2.5.1 Subcloning

The clones of 135 single-well cells from the first screening were subjected to a second assay (assay method as above). Based on the antibody titer and S/B ratio, 14 candidate clones were screened from the 135 candidate clones, and the assay results of the 14 clones are shown in Table 2. These 14 clones will be used for further screening.

### 2.6. Affinity Sorting

In order to screen the monoclonal antibody cell lines with relatively high affinity, 14 monoclonal antibody cell lines were subjected to affinity sorting. The results are shown in Table 3, only the three clones 5C12d2C8, 18F9G6D5 and 55F8C3B2 of the 14 monoclonal antibodies have higher confidence in the fitted curves. Among them, 5C12d2C8 had the highest affinity with a KD (M) of 4.55×10⁻⁹ and an Rmax (RU) of only 291.1 (Table 3), and the cell line of this clone was used as a candidate clone.

TABLE 2
Second round of subclonal screening (ELISA)

| Concentration | | | | | | | | | | | | |
|---|---|---|---|---|---|---|---|---|---|---|---|---|
| (ng/ml) | 1,000 | 500 | 250 | 125 | 62.5 | 31.25 | 15.62 | 7.81 | 3.9 | 1.95 | Blank | Titer |
| Dilution factor | 1:1000 | 1:2000 | 1:4000 | 1:8000 | 1:16000 | 1:32000 | 1:64000 | 1:128000 | 1:256000 | 1:512000 | Blank | |
| 26E7H12D8 | 2.825 | 2.658 | 2.609 | 2.497 | 2.195 | 1.986 | 1.572 | 1.224 | 0.835 | 0.54 | 0.063 | >1:512,00 |
| 45E4D9G3 | 2.742 | 2.66 | 2.644 | 2.551 | 2.372 | 2.169 | 1.798 | 1.391 | 0.999 | 0.759 | 0.063 | >1:512,000 |
| 12D6F3B6 | 2.689 | 2.659 | 2.639 | 2.574 | 2.433 | 2.166 | 1.862 | 1.393 | 0.945 | 0.668 | 0.063 | >1:512,000 |
| 5C12D2C8 | 2.685 | 2.619 | 2.579 | 2.478 | 2.273 | 1.972 | 1.563 | 1.127 | 0.718 | 0.436 | 0.054 | 1:512,000 |
| 15B2B3H1 | 2.878 | 2.808 | 2.786 | 2.705 | 2.596 | 2.449 | 2.201 | 1.849 | 1.43 | 1.003 | 0.054 | >1:512,000 |
| 20H9C1B7 | 2.868 | 2.794 | 2.712 | 2.659 | 2.553 | 2.385 | 2.079 | 1.749 | 3.337 | 0.932 | 0.054 | >1:512,000 |
| 74D7E4A6 | 2.77 | 2.738 | 2.723 | 2.624 | 2.492 | 2.218 | 1.8 | 1.352 | 0.899 | 0.616 | 0.063 | >1:512,000 |
| 60G12D8G5 | 2.854 | 2.727 | 2.607 | 2.5 | 2.272 | 1.997 | 1.552 | 1.067 | 8.724 | 0.464 | 0.054 | 1:512,000 |
| 64E3G5C10 | 2.861 | 2.701 | 2.609 | 2.393 | 2.117 | 1.744 | 1.315 | 0.945 | 0.599 | 0.374 | 0.054 | 1:512,000 |
| 55F8C3B2 | 2.745 | 2.668 | 2.593 | 2.466 | 2.218 | 1.956 | 1.544 | 1.063 | 0.74 | 0.481 | 0.063 | 1:512,000 |
| 68G5E5E7 | 2.849 | 2.73 | 2.768 | 2.655 | 2.519 | 2.358 | 2.033 | 1.653 | 1.202 | 0.833 | 0.063 | >1:512,000 |
| 18F9G6D5 | 2.708 | 2.676 | 2.565 | 2.525 | 2.327 | 2.081 | 1.719 | 1.305 | 0.745 | 0.35 | 0.054 | >1:512,000 |
| 18G5A5E2 | 2.64 | 2.625 | 2.52 | 2.435 | 2.287 | 2.023 | 1.642 | 1.218 | 0.794 | 0.491 | 0.054 | 1:512,000 |
| 60G12G2E2 | 2.881 | 2.762 | 2.673 | 2.585 | 2.391 | 2.174 | 1.756 | 1.38 | 0.908 | 0.559 | 0.054 | >1:512,000 |

The titer is the highest dilution with S/B (Signal/Blank) >=2.1, the OD450 in blank is the average of two technical replicates.

TABLE 3
Antigen-antibody binding affinity sorting

| Analyte | Kinetics Chi² | ka | kd | KD | Rmax |
|---|---|---|---|---|---|
| Solution | (RU²) | (1/Ms) | (1/s) | (M) | (RU) |
| 5C12D2C8 | 1.70E+00 | 5.06E+04 | 2.30E−04 | 4.55E−09 | 291.1 |
| 60G12D8G5 | 2.30E+00 | 1.43E+06 | 1.41E−02 | 9.90E−09 | 36.1 |
| 68G5E5E7 | 7.60E−01 | 4.59E+06 | 5.45E−02 | 1.19E−08 | 19.6 |
| 20H9C1B7 | 3.21E+00 | 2.36E+05 | 2.91E−03 | 1.24E−08 | 26.5 |
| 60G12G2E2 | 4.33E−01 | 2.69E+06 | 3.38E−02 | 1.26E−08 | 16 |
| 74D7E4A6 | 3.31E+00 | 2.00E+06 | 2.72E−02 | 1.36E−08 | 43.1 |
| 18F9G6D5 | 1.03E−02 | 8.95E+04 | 1.77E−03 | 1.98E−08 | 8.9 |
| 15B1B3H1 | 3.02E+01 | 2.86E+05 | 7.27E−03 | 2.54E−08 | 102.8 |
| 26E7H12D8 | 6.97E−01 | 1.44E+05 | 5.09E−03 | 3.53E−08 | 18.2 |
| 55F8C3B2 | 1.39E−02 | 9.68E+03 | 4.27E−04 | 4.41E−08 | 487.4 |
| 18G5A5E2 | N/A | N/A | N/A | N/A | N/A |
| 12D6F3B6 | N/A | N/A | N/A | N/A | N/A |
| 64E5G5C10 | N/A | N/A | N/A | N/A | N/A |
| 45E4D9G3 | N/A | N/A | N/A | N/A | N/A |

### Example 3 Antibody Immunogenicity Assay

### 3.1 Antigen Preparation

### 3.1.1: Construction of Vector and Preparation of Crude Protein

80 amino acid polypeptides containing the ion-conducting pore module target of the DIVS3 domain of Nav1.7 were synthesized, and a His fusion protein expression vector for prokaryotic expression was constructed, and inoculated into 2000 mL of LB liquid medium (Kan-resistant), and incubated at 37° C. with shaking overnight, and then allowed to reduce the incubation temperature to 30° C. when the OD600 was about 0.6; added with IPTG inducer to a final concentration of 0.1 mM, and continued to incubate at 30° C. for 8 h; centrifuged for 3 min to collect bacteria, which were resuspended in 50 mL of pre-cooled NTA-0 buffer, and subjected to ice-bath for 30 min, and then ultrasonically fragmented, and then centrifuged for 50 min at 4° C. to collect the precipitate (inclusion bodies). The precipitate was resuspended in 50 mL of NTA-0 buffer, and added with DTT to a final concentration of 1 mM; subjected to ultrasonication to promote the dissolution of heteroproteins, centrifuged at 4° C. for 10 min to remove the supernatant, to obtain the preparation solution of crude protein.

### 3.1.2 Protein Denaturation and Reconstitution

The protein solution was denatured by dilution with 2 times volume of 3M guanidine hydrochloride. The protein solution was put in a dialysis bag and the volume was concentrated to 50-100 mL with PEG20000 and dialyzed at 4° C. with PBS buffer overnight for denaturation, and then the protein was concentrated.

### 3.1.2 Protein Purification

The Ni-NTA column was prepared according to the manufacturer's instructions, and protein purification was performed according to the instructions.

### 3.2 Protein Western Blotting

(1) Making separation gel according to 12% separation gel formula;
(2) Mixing the corresponding antigen with the sample buffer 4:1 and denaturing at 95° C. for 5 min;
(3) Loading 4 μl of Marker and 50 μl of antigen to the lane respectively;
(4) Running at 90V to the end of the concentrated gel, and then running to ⅔ of the end of the separated gel at 120V;
(5) Taking out the gel to remove the concentrated gel, and putting the cut PVDF membrane in methanol for 2 min, and then equilibrating the gel, PVDF membrane, 6 cut filter paper and a supporting pad in a transfer buffer for 10 min;
(6) Assembling in the order of negative electrode→blackboard→supporting pad→3 filter papers→glue→membrane→3 filter papers→supporting pad→whiteboard→positive electrode, and driving out air bubbles with a glass rod in each layer;
(7) Filling the membrane transfer tank with membrane transfer buffer, transfering the membrane at 100V for 30 min, and put the membrane transfer tank into ice water;
(8) Taking out the membrane and staining it with Reichhorn red dye for 5 min, then rinsing it slightly with water to observe whether the membrane transfer is successful, and washing the Reichhorn red with washing solution after the appearance of bands;
(9) Moistening the membrane with blocking solution, then blocking it on shaking bed for 1 h, then washing with washing solution for 3 times, 5 min each;
(10) Cutting the membrane into 5 strips, taking 5 different hybridoma supernatants, diluting them with PBS at a ratio of 1:10 as primary antibody, and incubating at 4° C. overnight;
(11) On the next day, washing with washing solution for 3 times, 5 min each, adding HRP-sheep anti-mouse IgG diluted (1:5000) with washing solution and incubating for 1h at room temperature;
(12) Washing with washing solution for three times, 10 min each, dropring the prepared DAB working solution onto the membrane, and observing the results after color development in the dark for 8 min.

### 4. Western Blotting Results

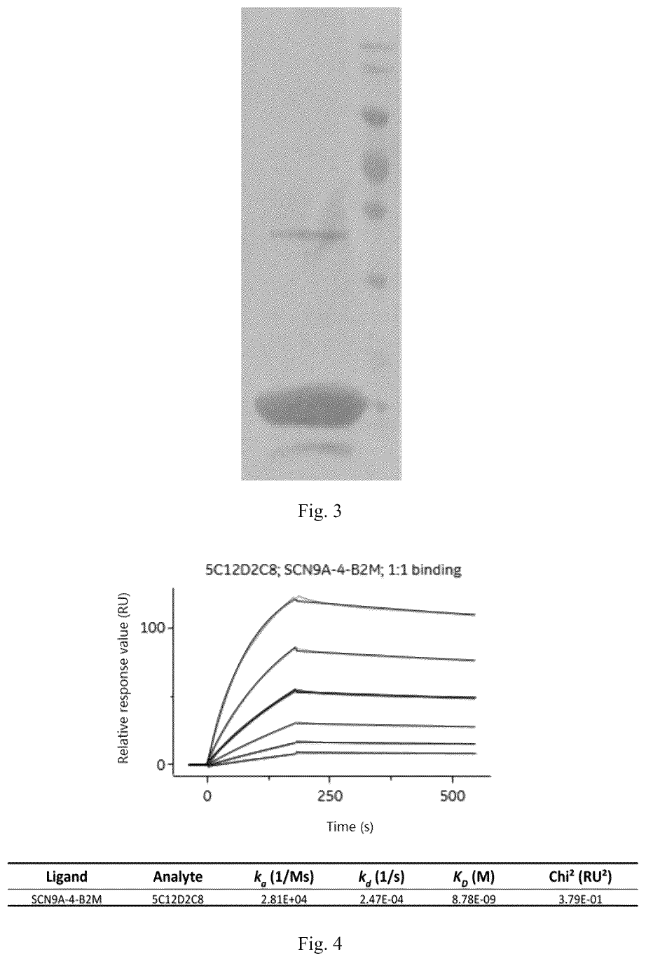

The monoclonal antibody 5C12D2C8 to Nav1.7 was hybridized with the covered target peptide antigen by Western Blotting, and the results are shown in FIG. 3. The results showed that 5C12D2C8 can recognize the target polypeptide antigen with antigen-antibody immunoreaction, proving that 5C12D2C8 is able to recognize the target and has good specificity.

### Example 4 Sequencing of Antibodies

To determine the monoclonal antibody sequence, monoclonal 5C12D2C8 was sequenced. Total RNA was isolated from hybridoma cells according to the technical manual for TRIzol reagent. Total RNA was then reverse transcribed into cDNA using isoform-specific antisense primers or universal primers, following the PrimeScript TM First Strand cDNA Synthesis Kit Technical Manual. Antibody fragments for VH and VL were amplified according to GenScript's Rapid Amplification of cDNA Ends (RACE) standard operating procedure (SOP) method. The amplified antibody fragments were cloned into standard cloning vectors, respectively. Colony PCR was performed to screen the clones with insert fragments of the correct size. At least 5 colonies with correctly sized insert fragments were sequenced. The sequences of the different clones were compared to determine the common sequence of these clones.

Finally, the DNA sequence of VH was determined as shown in SEQ ID NO: 10; the DNA sequence of VL was determined as shown in SEQ ID NO: 11.

The DNA sequence of the heavy chain variable region (VH) of monoclonal antibody 5C12D2C8 is shown in SEQ ID NO: 10; the DNA sequence of the light chain variable region (VL) is shown in SEQ ID NO: 11. VH and VL contain three complementary determining regions (CDRs) respectively, each CDR positions as the following: the dashed line underlined is the guide sequence (signal peptide), the solid line underlined is the CDR sequence, and the boldface is the framework region (FR).

Heavy chain:

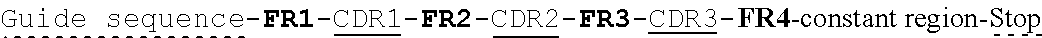

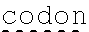

Heavy chain variable region (VH):
(SEQ ID NO: 10)

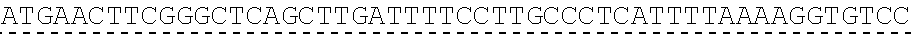

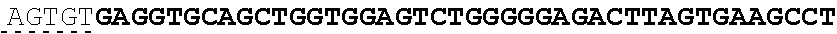

GGAGGGTCCCTGAAACTCTCCTGTGCAGCCTCTGGATTCACTTTCAG

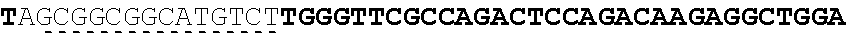

GTGGGTCGCAACCATTAGTAATGGTGGTGGTGAGACCTACTATGAGGACA
GTGTGAAGGGGCGATTCACCATCTCCAGAGACAATGCCAAGAACACCC
TGTACCTACAAATGAACAGTCTGAAGTCTGAGGACACAGCCAT

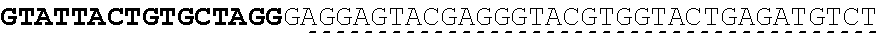

GGGGCGCAGGGACCACGGTCACCGTCTCCTCA
Heavy chain constant region:
GCCAAAACGACACCCCCATCTGTCTATCCACTGGCCCCTGGATCTGCTGCC
C
AAACTAACTCCATGGTGACCCTGGGATGCCTGGTCAAGGGCTATTTCCCTG
AGCCAGTGACAGTGACCTGGAACTCTGGATCCCTGTCCAGCCGTGTGCAC
ACCTTCCCACCTCTCCTCCAGTCTGACCTCTAC
ACTCTGAGCAGCTCAGTGACTGTCCCCTCCAGCACCTGGCCCAGCGAGAC
CGTCACCTGCAACGTTG
CCCACCCGGCCAGCAGCACCAAGGTGGACAAGAAAATTGTGCOCAGGGA
TIGTGGTTGTAAGCCTTG
CATATGTACAGTCCCAGAAGTATCATCTGTCTTCATCTTCCCCCCAAAGCCC
AAGGATGTGCTCACC
ATTACTCTGACTCCTAAGGTCACGTGTGTTGTGGTAGACATCAGCAAGGAT
GATCCCGAGGTCCAGT
TCAGCTGGTTTGTAGATGATGTGGAGGTGCACACAGCTCAGACGCAACCC
CGGGAGGAGCAGTTCAA
CAGCACTTTCCGCTCAGTCAGTGAACTTCCCATCATGCACCAGGACTGGCT
CAATGGCAAGGAGTTC
AAATGCAGGGTCAACAGTGCAGCTTTCCCTGCCCCCATCGAGAAAACCAT
CTCCAAAACCAAAGGCA
GACCGAAGGCTCCACAGGTGTACACCATTCCACCTCCCAAGGAGCAGATG
GCCAAGGATAAAGTCAG
TCTGACCTGCATGATAACAGACTTCTTCCCTGAAGACATTACTGTGGAGTG
GCAGTGGAATGGGCAG
CCAGOGGAGAACTACAAGAACACTCAGCCCATCATGGACACAGATGGCTC
TTACTTCGTCTACAGCA
AGCTCAATGTGCAGAAGAGCAACTGGGAGGCAGGAAATACTTTCACCTGC
TCTGTGTTACATGAGGG
CCTGCACAACCACCATACTGAGAAGAGCCTCTCCCACTCTCCTOGTAAATG
A
Light chain:

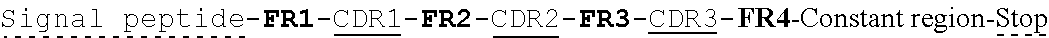

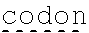

Light chain variable region (VL):
(SEQ ID NO: 11)

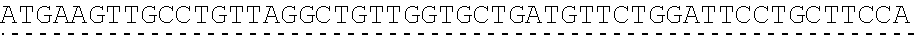

GCAGTGATGTTTTGATGACCCAAACTCCACTCTCCCTGCCTGTCAGTC

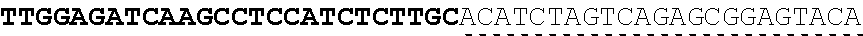

TAGTAATGGAAACACCGAGTTAGAATGGTACCTGCAGAAACCAGGCCA
GTCT
CCAAAGCTCCTGATCTACAAAGTTTCCAACCGAACATCTGGGGTCCCAG
ACAGGTTCAGTGGCAGTG
GATCAGGGACAGATTTCACACTCAAGATCAGCAGAGTGGAGGCTGAG
GATCTGGGAGTTTATTACTGCGGCCAAGGTTCACATGTTGGCCGGACGT
TCGGTGGAGGCACCAAGCTGGAAATCAAA
Light chain constant region:
CGGGCTGATGCTGCACCAACTGTATCCATCTTCCCACCATCCAGTGAGCAG
TTAACATCTGGAGGTGCCTCAGTCGTGTGCTTCTTGAACAACTTCTACCCC
AAAGACATCAATGTCAAGTGGAAGATTGATGGCAGIGAACG
ACAAAATGGCGTCCTGAACAGTTGGACTGATCAGGACAGCAAAGACAGC
ACCTACAGCATGAGCAOC
ACCCTCACGTTGACCAAGGACGAGTATGAACGACATAACAGCTATACCTGT
GAGGCCACTCACAAGA
CATCAACTTCACCCATTOTCAAGAGCTTCAACAGGAATGAGTGTTAG

The amino acid sequence was deduced from the DNA sequence. The amino acid sequence of VH is shown in SEQ ID NO: 7 and the amino acid sequence of VL is shown in SEQ ID NO: 8.

Heavy chain:

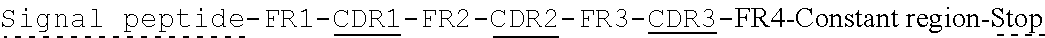

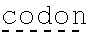

Heavy chain variable region (VH):
(SEQ ID NO:7)

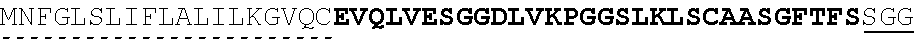

MSWVRQTPDKRLEWVATISNGGGETYYEDSVKGRFTISRDNAKNTLYLQ
MNSLKSEDTAMYYCAREEYEGTWYEDVWGAGTTVTVSS
Constant region
AKTTPPSVYPLAPGSAAQTNSMVTLGCLVKGYFPEPVTVTWNSGSLSSGVHT
FPAVLQSDLYTLSSSVTVPSSTWPSETVTCNVAHPASSTKVDKKIVPRDCGCKP
CICTVPEVSSVFIFPPKPKDVLT
ITLTPKVTCVVVDISKDDPEVQFSWFVDDVEVHTAQTQPREEQFENSTERSVSE
LPIMHQDWLNGKEF
KCRVNSAAFPAPIEKTISKTKGRPKAPQVYTIPPPKEQMAKDKVSLTCMITDFF
PEDITVEWQWNGQ
PAENYKNTQPIMDTDGSYFVYSKLNVOKSNWEAGNTFTCSVLHEGLHNHHT
EKSLSHSPGK-
Light chain:

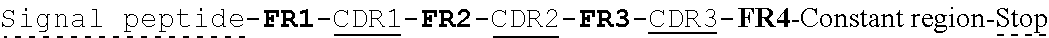

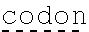

Light chain variable region (VL)
(SEQ ID NO:8)

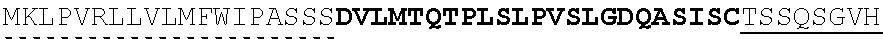

SNGNTELEWYLQKPGQSPKLLIYKVSNRTSGVPDRFSGSGSGTDFTLKL

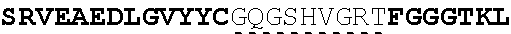

KEI
Constant region
RADAAPTVSIFPPSSEQLTSGGASVVCFLNNFYPKDINVKWKIDGSERQNG
VLNSWTDQDSK
DSTYSMSSTLTLTKDEYERHNSYTCEATHKTSTSPIVKSFNRNEC-

It was deduced that the RNA sequence encoding VH is shown in SEQ ID NO: 12 and the RNA sequence encoding VL is shown in SEQ ID NO: 13.

### Example 5 Determination of Affinity of 5C12D2C8 Antibody to Antigen

The antigen-antibody dissociation rate equilibrium constant (KD) reflects the affinity between the antibody and the antigen, and the lower the KD value, the higher the affinity. Surface plasmon resonance (SPR) was used to determine the antigen-antibody dissociation rate equilibrium constant of a monoclonal antibody, to evaluate the affinity of the monoclonal antibody to the antigen.

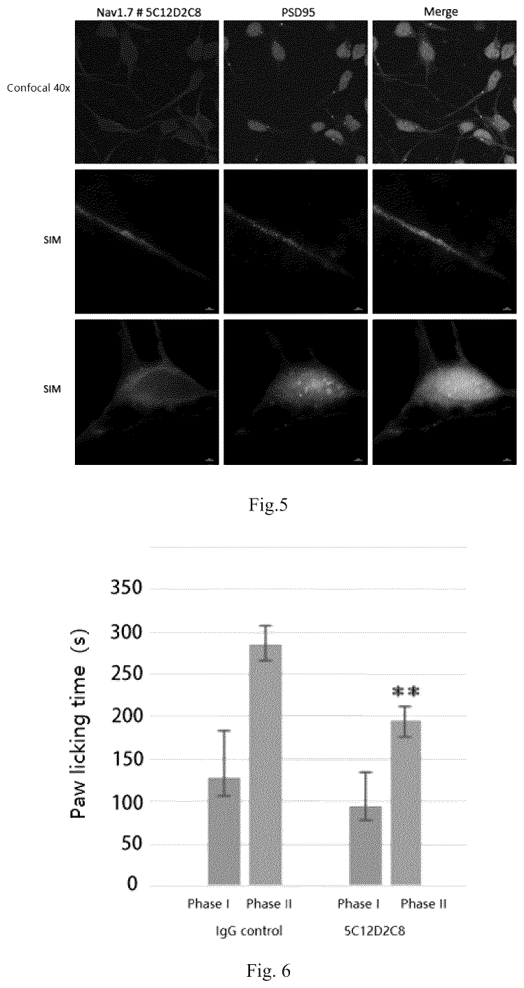

The binding curves of monoclonal antibody 5C12D2C8 were determined by SPR at different concentrations of 12.5, 25, 50, 100, 200 and 400 nM. The results are shown in FIG. 5. The dissociation constant Kd (l/s) of monoclonal antibody 5C12D2C8 to antigen was 2.47×10⁻⁴ and the equilibrium dissociation constant KD (M) was 8.78×10⁻⁹ (FIG. 4).

### Example 6 Specificity of 5C12D2C8 Antibody in Human Peripheral Nerve Cells

Since nerve cells are highly differentiated, in order to test the specificity of 5C12D2C8 in human peripheral nerve cells, iPS-induced human peripheral nerve cells were used for validation. Before performing the test, the type of iPS-induced neuronal cells was confirmed, molecular markers specific for peripheral nerve tissues were used to identify whether the induced peripheral nerve well developed. Three molecular markers specifically expressed in neuronal cells, PSD95, were used to localize human nerve tissues. PSD95 is encoded by the DLG4 gene, a member of the membrane-associated guanylate kinase (MAGUK) family, which interacts with PSD93 at postsynaptic sites and is specifically expressed in nerve tissues. Therefore, the PSD95 antibody can be used as a molecular marker (control) to study the specificity and distribution of antibody expression in human peripheral nerve cells.

### 6.1 iPS Induction of Human Peripheral Neurons

6.1.1: T25 culture flasks were coated with 2 μg/cm² of laminin (SIGMA); human-derived nerve stem cells (iRegene Therapeutics) were inoculated into 2 coated T25 culture flasks at 2.5×10⁶ cells per flask in serum-free, animal origin-free FP neural stem cell medium (iRegene Therapeutics) where 10 UM of inhibitor Y-27632 was added. After inoculation, the T25 culture flasks were incubated overnight at 37° C., 5% CO₂.
6.1.2: The following day, the FP neural stem cell medium containing Y-27632 was removed and replaced with serum-free, animal origin-free peripheral neuronal directed differentiation medium (iRegene Therapeutics), and this medium was then used until the end of the experiment. The medium was then changed every other day until day 14; cell morphology changes were recorded in between.
6.1.3: Double-coated 15 mm optical slides (Deckglaser) were used to prepare cell climbing slides. 15 mm optical slides were placed in 24-well plates (1 slide/well). The slides were submerged with 50 μg/mL poly-L-lysine solution and incubated at 37° C., 5% CO₂ for no less than 2 hours. The poly-L-lysine was removed and washed three times with sterilized water. The slides were submerged using 5 μg/mL of Laminin solution and incubated at 37° C., 5% CO₂ for not less than 3 hours, after which they were washed well with PBS.
6.1.4: Cells in T25 culture flasks were digested using Accutase Cell Digest (Invitrogen) and cells were inoculated onto the coated 15 mm optical slides (Deckglaser) at 1×10⁵ cell/well. The medium was serum-free, animal origin-free neuronal directed differentiation medium (iRegene Therapeutics), and the medium was changed every other day until day 21.

### 6.2 Fluorescence Immunoassay

6.2.1: The medium for cell differentiation was removed from the 24-well microtiter plates and the culture wells were washed using DPBS.
6.2.2: Cells were fixed using 4% paraformaldehyde for 40 minutes at room temperature and washed twice with DPBS buffer; then treated with 0.1% Triton X-100 for 5 minutes and then washed twice with DPBS buffer. The cells were then incubated with DPBS buffer containing 10% horse serum and 0.1% Triton X-100 at 4° C. overnight; finally, the antibody diluted with DPBS buffer was added and incubated for 2 hours at 37° C. and washed three times with DPBS buffer; Afterwards, the cells were incubated with fluorescent secondary antibody (1:1000) corresponding to the primary antibody, and the secondary antibody was removed after 45 min and washed three times with DPBS buffer; nuclear staining was performed using 300 nM DAPI for 2 minutes at room temperature; optical cell climbing slides were prepared by washing the seals three times with sterilized water; and read with an OLYMPUS FV3000 laser confocal microscope or a NIKON N-SIM high-resolution microscope.

### 6.3 Localization Results of Human Peripheral Nerve Cells

The cell type analysis of iPS-induced peripheral nerve cells was carried out using the peripheral nerve cell-specific molecular marker PSD95. The results showed that iPS-induced peripheral nerve cells can bind to the specific molecular marker, which proves that iPS-induced nerve cells belong to peripheral nerve cells, and can be used for cell-specific analysis of antibodies. The distribution of monoclonal antibody 5C12D2C8 in iPS-induced human peripheral nerve cells is shown in FIG. 5. Monoclonal antibody 5C12D2C8 showed stronger fluorescence signal on axons of peripheral nerve cells, compared with the control PSD95. The results showed that Nav1.7 channel monoclonal antibody 5C12D2C8 co-localized with molecular markers specific to axonal molecules, indicating that 5C12D2C8 has good peripheral nerve cell specificity and is aligned in dots on axons of peripheral nerve cells.

### Example 7 Analgesic Effect of 5C12D2C8 Antibody in Wild-type Mice

When injury receptors are stimulated, action potentials are generated and transmitted through peripheral nerves to the spinal cord and then to the brain to feel pain, and the Nav1.7 voltage-gated sodium channel plays a crucial role in the generation and transmission of action potentials. We hypothesize that monoclonal antibody drugs specifically bind to the ion-conducting pore of the voltage-gated sodium channel Nav1.7, which may block electrical signaling and thus achieve analgesia.

In order to validate and detect the analgesic effect of the monoclonal antibody 5C12D2C8, the formalin inflammatory pain model was used in this study. Since the pain caused by formalin experiments is more oriented to tension pain and elicits a state very similar to clinical pain, which is widely used to determine the pain perception of animals. After injection of formalin, the response behavior of animals to injurious stimuli are presented in 2 periods, the first period arises immediately after injection and lasts for about 10 min, and the second period appears about 15 min after injection and lasts for about 45 min, and thus the 2 periods are named as phase I and phase II, respectively. It is generally believed that phase I pain is caused by the direct stimulation of injury receptors by formalin, which leads to the excitation of C fibers and thus produces pain; while phase II pain is caused by inflammation-induced release of neurotransmitters such as prostaglandins, histamine, and 5-hydroxytryptamine, which excites the nociceptive nerve to produce pain. It is generally believed that phase II is more responsive to the effects of the drugs.

### 7.1 Experimental Methods

### 7.1.1: Experimental Apparatus

Mouse tail injection fixator (YLS-Q9G) was purchased from Shanghai Bio-will Co., Ltd. and 50 μl micro syringe and 1 ml human insulin syringe were purchased from Wuhan Qinzhijie Biological Co., Ltd.

7.1.2: Experimental Animal 25-35 g SPF grade KM mice were purchased from Hubei Animal Experiment and Research Center; KM mice were purchased to acclimatize in the laboratory for at least 2 days, with an ambient temperature of 23±1° C., and were given adequate water as well as food, 5-8 mice per cage. The mice were acclimatized in transparent cages for 30 minutes prior to the experiment. No food or water was allowed during the experiment. A timer was used to record the results, and at the end of the experiment the animals were anesthetized and euthanized by placing them in ether vials.

### 7.1.3: Drug Treatment Method

Each mouse was taken out from the transparent cage and placed on the tabletop and put into a fixator by pulling its tail. Then the head was fixed with a stopper so that its tail extended out of the bottom of the fixator. The fixator was placed on the tail injection auxiliary table, and the tail of the mouse was obsereved under the auxiliary lamp. After the lateral blood vessels were observed, 200 μl of monoclonal antibody drug was injected into the lateral blood vessels by tail vein injection, after the needle was pulled out, the wound was pressed for 20 s to stop bleeding, and then the mouse was placed into the transparent cage. After 30 min, 20 μl of freshly prepared 5% formalin solution was injected into the plantar surface of the mouse using a microsyringe, and then the mouse was immediately placed in the transparent cage for observation. A timer was used to record the time(s) for the mouse to lick the injected paw over a 45-min period, and the data were recorded at 5-min intervals, by recording the time of paw licking and paw retraction during each 5-min period, for a total of 45 min. Phase I acute pain (0-10 min) and phase II persistent pain (10-45 min) were statistically analyzed, respectively, using murine IgG control antibody with equal volume and concentration as the control group.

### 7.1.4: Data Processing

The data were subjected to two-tailed T-test to determine if there was a significant difference. In the same dose experiment, the data of the experimental group was subjected to two-tailed T-test with both PBS control and IgG control; in different dose experiments, the data of the drug experimental group were compared with the PBS control group as well as equal dose IgG control group and also between different doses of the same drug. Significant differences were indicated by *, where * indicated p<0.05 and ** indicated p<0.01.

### 7.2 Experimental Results

The analgesic effect of monoclonal antibody 5C12D2C8 at a dose of 25 mg/kg is shown in FIG. 6. IgG negative antibody control (IgG) showed a significant increase in paw licking time in phase II over phase I at the same dose. For the mice injected with monoclonal antibody 5C12D2C8, the paw licking time of in phase II was significantly different (P<0.01) from that in phase I and in control group after administration. The analgesic effect of monoclonal antibody 5C12D2C8 in phase II was reduced by 32% compared to the negative antibody control (IgG) (FIG. 6).

Sequence Listing:
HCDR 1:
(SEQ ID NO: 1)
SGGMS
HCDR2:
(SEQ ID NO: 2)
TISNGGGETYYEDSVKG
HCDR 3:
(SEQ ID NO: 3)
EEYEGTWYEDV
LCDR 1:
(SEQ ID NO: 4)
TSSQSGVHSNGNTELE
LCDR2:
(SEQ ID NO: 5)
KVSNRTS
LCDR 3:
(SEQ ID NO: 6)
GQGSHVGRT
VH:
(SEQ ID NO: 7)
MNFGLSLIFLALILKGVQCEVQLVESGGDLVKPGGSLKLSCAASG
FTFSSGGMSWVRQTPDKRLEWATISNGGGETYYEDSVKGRFTISR
DNAKNTLYLQMNSLKSEDTAMYYCAREEYEGTWYEDVWGAGTVTV
SS
Heavy chain:
(SEQ ID NO: 14)
MNFGLSLIFLALILKGVQCEVQLVESGGDLVKPGGSLKLSCAASG
FTFSSGGMSWVRQTPDKRLEWATISNGGGETYYEDSVKGRFTISR
DNAKNTLYLQMNSLKSEDTAMYYCAREEYEGTWYEDVWGAGTAKT
TPPSVYPLAPGSAAQTNSMVTLGCLVKGYFPEPVTVTWNSGSLSS
GVHTFPAVLQSDLYTLSSSVTVPSSTWPSETVTCNVAHPASSTKV
DKKIVPRDCGCKPCICTVPEVSSVFIFPPKPKDVLTITLTPKVTC
VVVDISKDDPEVQFSWFVDDVEVHTAQTQPREEQFNSTFRSVSEL
PIMHQDWLNGKEFKCRVNSAAFPAPIEKTISKTKGRPKAPQVYTI
PPPKEQMAKDKVSLTCMITDFFPEDITVEWQWNGQPAENYKNTQP
IMDTDGSYFVYSKLNVQKSNWEAGNTFTCSVLHEGLHNHHTEKSL
SHSPGK-
VL:
(SEQ ID NO: 8)
MKLPVRLLVLMFWIPASSSDVLMTQTPLSLPVSLGDQASISCTSS
QSGVHSNGNTELEWYLQKPGQPKLLIYKVSNRTSGVPDRFSGSGS
GTDFTLKISRVEAEDLGVYYCGQGSHVGRTFGGGTKLEI
Light chain:
(SEQ ID NO: 15)
MKLPVRLLVLMFWIPASSSDVLMTQTPLSLPVSLGDQASISCTSS
QSGVHSNGNTELEWYLQKPGQPKLLIYKVSNRTSGVPDRFSGSGS
GTDFTLKISRVEAEDLGVYYCGQGSHVGRTFGGGTKLEIKRAAAP
TVSIFPPSSEQLTSGGASVVCFLNNFYPKDINVKWKIDGSERQNG
VLNSWTDQDSKDSTYSMSSTLTLTKDEYERHNSYTCEATHKTSTS
PIVKSFNRNEC-
Antigen peptide:
(SEQ ID NO: 9)
DSVNVDKQPKYEYS
VH nucleotide sequence:
(SEQ ID NO: 10)
atgaacttcgggctcagcttgattttccttgccctcattttaaaa
ggtgtccagtgtgaggtgcagctggtggagtctgggggagactta
gtgaagcctggagggtccctgaaactctcctgtgcagcctctgga
ttcactttcagtagcggcggcatgtcttgggttcgccagactcca
gacaagaggctggagtgggtcgcaaccattagtaatggtggtggt
gagacctactatgaggacagtgtgaaggggcgattcaccatctcc
agagacaatgccaagaacaccctgtacctacaaatgaacagtctg
aagtctgaggacacagccatgtattactgtgctagggaggagtac
gagggtacgtggtactgagatgtctggggcgcagggaccacggtc
accgtctcctca
Heavy chain nucleotide sequence:
(SEQ ID NO: 16)
atgaacttcgggctcagcttgattttccttgccctcattttaaaa
ggtgtccagtgtgaggtgcagctggtggagtctgggggagactta
gtgaagcctggagggtccctgaaactctcctgtgcagcctctgga
ttcactttcagtagcggcggcatgtcttgggttcgccagactcca
gacaagaggctggagtgggtcgcaaccattagtaatggtggtggt
gagacctactatgaggacagtgtgaaggggcgattcaccatctcc
agagacaatgccaagaacaccctgtacctacaaatgaacagtctg
aagtctgaggacacagccatgtattactgtgctagggaggagtac
gagggtacgtggtactgagatgtctggggcgcagggaccacggtc
accgtctcctcagccaaaacgacacccccatctgtctatccactg
gcccctggatctgctgcccaaactaactccatggtgaccctggga
tgcctggtcaagggctatttccctgagccagtgacagtgacctgg
aactctggatccctgtccagcggtgtgcacaccttcccagctgtc
ctgcagtctgacctctacactctgagcagctcagtgactgtcccc
tccagcacctggcccagcgagaccgtcacctgcaacgttgcccac
ccggccagcagcaccaaggtggacaagaaaattgtgcccagggat
tgtggttgtaagccttgcatatgtacagtcccagaagtatcatct
gtcttcatcttccccccaaagcccaaggatgtgctcaccattact
ctgactcctaaggtcacgtgtgttgtggtagacatcagcaaggat
gatcccgaggtccagttcagctggtttgtagatgatgtggaggtg
cacacagctcagacgcaaccccgggaggagcagttcaacagcact
ttccgctcagtcagtgaacttcccatcatgcaccaggactggctc
aatggcaaggagttcaaatgcagggtcaacagtgcagctttccct
gcccccategagaaaaccatctccaaaaccaaaggcagaccgaag
gctccacaggtgtacaccattccacctcccaaggagcagatggcc
aaggataaagtcagtctgacctgcatgataacagacttcttccct
gaagacattactgtggagtggcagtggaatgggcagccagcggag
aactacaagaacactcagcccatcatggacacagatggctcttac
ttcgtctacagcaagctcaatgtgcagaagagcaactgggaggca
ggaaatactttcacctgctctgtgttacatgagggcctgcacaac
caccatactgagaagagcctctcccactctcctggtaaatga
VL nucleotide sequence:
(SEQ ID NO: 11)
atgaagttgcctgttaggctgttggtgctgatgttctggattcct
gcttccagcagtgatgttttgatgacccaaactccactctccctg
cctgtcagtcttggagatcaagcctccatctcttgcacatctagt
cagagcggagtacatagtaatggaaacaccgagttagaatggtac
ctgcagaaaccaggccagtctccaaagctcctgatctacaaagtt
tccaaccgaacatctggggtcccagacaggttcagtggcagtgga
tcagggacagatttcacactcaagatcagcagagtggaggctgag
gatctgggagtttattactgcggccaaggttcacatgttggccgg
acgttcggtggaggcaccaagctggaaatcaaa
Light chain nucleotide sequence:
(SEQ ID NO: 17)
atgaagttgcctgttaggctgttggtgctgatgttctggattcct
gcttccagcagtgatgttttgatgacccaaactccactctccctg
cctgtcagtcttggagatcaagcctccatctcttgcacatctagt
cagagcggagtacatagtaatggaaacaccgagttagaatggtac
ctgcagaaaccaggccagtctccaaagctcctgatctacaaagtt
tccaaccgaacatctggggtcccagacaggttcagtggcagtgga
tcagggacagatttcacactcaagatcagcagagtggaggctgag
gatctgggagtttattactgcggccaaggttcacatgttggccgg
acgttcggtggaggcaccaagctggaaatcaaacgggctgatgct
gcaccaactgtatccatcttcccaccatccagtgagcagttaaca
tctggaggtgcctcagtcgtgtgcttcttgaacaacttctacccc
aaagacatcaatgtcaagtggaagattgatggcagtgaacgacaa
aatggcgtcctgaacagttggactgatcaggacagcaaagacagc
acctacagcatgagcagcaccctcacgttgaccaaggacgagtat
gaacgacataacagctatacctgtgaggccactcacaagacatca
acttcacccattgtcaagagcttcaacaggaatgagtgttag
RNA sequences:
(SEQ ID NO: 12)
AUGAACUUCGGGCUCAGCUUGAUUUUCCUUGCCCUCAUUUUAAAA
GGUGUCCAGUGUGAGGUGCAGCUGGUGGAGUCUGGGGGAGACUUA
GUGAAGCCUGGAGGGUCCCUGAAACUCUCCUGUGCAGCCUCUGGA
UUCACUUUCAGUAGCGGCGGCAUGUCUUGGGUUCGCCAGACUCCA
GACAAGAGGCUGGAGUGGGUCGCAACCAUUAGUAAUGGUGGUGGU
GAGACCUACUAUGAGGACAGUGUGAAGGGGCGAUUCACCAUCUCC
AGAGACAAUGCCAAGAACACCCUGUACCUACAAAUGAACAGUCUG
AAGUCUGAGGACACAGCCAUGUAUUACUGUGCUAGGGAGGAGUAC
GAGGGUACGUGGUACUGAGAUGUCUGGGGCGCAGGGACCACGGUC
ACCGUCUCCUCA
(SEQ ID NO: 13)
AUGAAGUUGCCUGUUAGGCUGUUGGUGCUGAUGUUCUGGAUUCCU
GCUUCCAGCAGUGAUGUUUUGAUGACCCAAACUCCACUCUCCCUG
CCUGUCAGUCUUGGAGAUCAAGCCUCCAUCUCUUGCACAUCUAGU
CAGAGCGGAGUACAUAGUAAUGGAAACACCGAGUUAGAAUGGUAC
CUGCAGAAACCAGGCCAGUCUCCAAAGCUCCUGAUCUACAAAGUU
UCCAACCGAACAUCUGGGGUCCCAGACAGGUUCAGUGGCAGUGGA
UCAGGGACAGAUUUCACACUCAAGAUCAGCAGAGUGGAGGCUGAG
GAUCUGGGAGUUUAUUACUGCGGCCAAGGUUCACAUGUUGGCCGG
ACGUUCGGUGGAGGCACCAAGCUGGAAAUCAAA

What is claimed is:

1. An antibody or antibody fragment thereof that specifically binds to voltage-gated sodium channel α subunit Nav1.7, wherein the antibody or antibody fragment specifically binds to an ion-conducting pore module of DIVS3 domain of the voltage-gated sodium channel α subunit.

2. The antibody or antibody fragment thereof according to claim 1, characterized in that the antibody or antibody fragment comprises:
heavy chain complementary determining regions HCDR1, HCDR2, HCDR3, the HCDR1 having the amino acid sequence as shown in SEQ ID NO. 1, the HCDR2 having the amino acid sequence as shown in SEQ ID NO. 2, and the HCDR3 having the amino acid sequence as shown in SEQ ID NO. 3; and
light chain complementary decision regions LCDR1, LCDR2, LCDR3, the LCDR1 having the amino acid sequence as shown in SEQ ID NO. 4, the LCDR2 having the amino acid sequence as shown in SEQ ID NO. 5, and the LCDR3 having the amino acid sequence as shown in SEQ ID NO. 6.

3. The antibody or an antibody fragment thereof according to any one of claims 1-2, characterized in that the antibody or antibody fragment comprises a heavy chain variable region as shown in SEQ ID NO.7 and/or a light chain variable region as shown in SEQ ID NO.8.

4. The antibody or antibody fragment thereof according to any one of claims 1-3, characterized in that the antibody further comprises an antibody constant region.

5. The antibody or an antibody fragment thereof according to any one of claims 1-4, the antibody or antibody fragment comprises a heavy chain as shown in SEQ ID NO. 14 and/or a light chain as shown in SEQ ID NO. 15.

6. The antibody or antibody fragment thereof according to claim 1, characterized in that the antibody or antibody fragment is in a structural form selected from the group consisting of a full antibody, Fab, F(ab′)₂, dsFv, scFv, a diabody, a minibody, a bispecific antibody, a multi-specific antibody, a chimeric antibody, and a CDR-grafted antibody.

7. The antibody or antibody fragment thereof according to claim 1, characterized in that the antibody is a monoclonal antibody.

8. The antibody or antibody fragment thereof according to claim 7, characterized in that the antibody is a humanized antibody.

9. A polypeptide that specifically binds to the antibody or antibody fragment according to claim 1, wherein the polypeptide has an amino acid sequence as shown in SEQ ID NO.9.

10. An isolated nucleotide encoding the antibody or antibody fragment that specifically binds to voltage-gated sodium channel α subunit Nav1.7 according to any one of claims 1-8.

11. An expression vector containing the nucleotide according to claim 10.

12. A host cell containing the nucleotide according to claim 10 or the expression vector according to claim 11.

13. A method of preparing the antibody or antibody fragment that specifically binds to the voltage-gated sodium channel α subunit Nav1.7 according to claim 1, wherein the method comprising the steps of:
(a) culturing the host cell according to claim 12 under expression conditions so as to express the antibody or antibody fragment that specifically binds to the voltage-gated sodium channel α subunit Nav1.7;
(b) isolating and purifying the antibody or antibody fragment that specifically binds to the voltage-gated sodium channel α subunit Nav1.7.

14. A pharmaceutical composition, comprising the antibody or antibody fragment according to anyone of claims 1-8.

15. Use of the antibody or antibody fragment according to claims 1-8 or of the pharmaceutical composition according to claim 14 for the manufacture of a medicament for the treatment of pain-related diseases.
